(19) United States
(12) Patent Application Publication
(10) Pub. No.: US 2020/0406061 A1
(43) Pub. Date: Dec. 31, 2020

(54) EMPLOYING SPECTRAL (MUTLI-ENERGY) IMAGE DATA WITH IMAGE GUIDED APPLICATIONS

(71) Applicant: KONINKLIJKE PHILIPS N.V., Eindhoven (NL)

(72) Inventors: Douglas B. MCKNIGHT, Chardon, OH (US); Chuanyong BAI, Solon, OH (US)

(73) Assignee: Koninklijke Philips N.V., Eindhoven (NL)

(21) Appl. No.: 16/646,666

(22) PCT Filed: Sep. 7, 2018
(86) PCT No.: PCT/EP2018/074145

§ 371 Date: Mar. 12, 2020

Related U.S. Application Data
(60) Provisional application No. 62/557,182, filed on Sep. 12, 2017.

(51) Int. Cl. A61N 5/10; A61B 6/03; A61B 6/00; A61B 34/30; A61B 18/14

(52) U.S. Cl. CPC A61N 5/1039; A61B 6/032; A61B 6/037; A61B 6/4241; A61B 2018/00577; A61B 6/50; A61B 34/30; A61B 18/14; A61B 6/463

(57) ABSTRACT

A system (1) includes a device (12, 116 or 118) with memory with spectral volumetric image data generated by a spectrally configured computed tomography scanner including a radiation source and a radiation detector and an image guided system (14) configured to employ the spectral volumetric image data for an image guided procedure. A computer readable medium is encoded with computer executable instructions, where the computer executable instructions, when executed by a processor, causes the processor to: obtain spectral volumetric image data generated by a spectrally configured computed tomography scanner including a radiation source and a radiation detector, and employ the spectral volumetric image data for an image guided procedure. A method includes receiving spectral volumetric image data generated by a spectrally configured computed tomography scanner including a radiation source and a radiation detector, and utilizing he spectral volumetric image data for an image guided procedure.

### FIELD OF THE INVENTION

The following generally relates to employing spectral (multi-energy) image data with image guided applications (e.g., ablation, robotic, radiation therapy, single photon emission computed tomography (SPECT), positron emission computed tomography (PET), and is described herein with particular application to a computed tomography (CT) scanner configured to generate spectral (multi-energy) volumetric image data and/or images.

### BACKGROUND OF THE INVENTION

A non-spectral computed tomography (CT) scanner generally includes a polychromatic x-ray tube mounted on a rotatable gantry opposite one or more rows of non-energy resolving detectors. The x-ray tube rotates around an examination region located between the x-ray tube and the one or more rows of detectors and emits polychromatic radiation that traverses the examination region and a subject and/or object disposed in the examination region. The one or more rows of detectors detect radiation that traverses the examination region and generate a signal (projection data) indicative of the examination region and the subject and/or object disposed therein. The projection data is proportional to the energy fluence integrated over the energy spectrum.

The projection data is reconstructed to generate volumetric image data by means of a computer, which can be used to generate one or more images. The volumetric image data is a weighted average of the linear attenuation coefficients of the subject and/or object within the spectrum of the polychromatic X-ray beam. The resulting image(s) includes pixels that are represented in terms of gray scale values corresponding to relative radiodensity. Such information reflects the attenuation characteristics of the scanned subject and/or object, and generally shows structure such as anatomical structures within a patient, physical structures within an inanimate object, and the like. These images are dependent on the X-ray source and properties of the photon detectors.

The volumetric image data has been used for diagnosis, image guided surgery, image guided ablation, image guided radiation therapy planning, CT-based attenuation correction in PET/CT and SPECT/CT, and/or other applications. However, the volumetric image data is not optimal for all applications. For example, the volumetric image data can have low tumor to soft tissue contrast and thus has limited use for the detection/identification and delineation of tumors for diagnosis and image guided applications, and can lead to suboptimal and large inter-operator variance of planning. The quantitative value in the Hounsfield unit (HU) is only for a value at an approximated effective energy (e.g., an effective kVp).

Furthermore, the electron density information derived from the volumetric image data can have a large error when there are high-Z materials. As such, dose simulation, planning, and/or calculation using such volumetric image data based on the electron density information derived therefrom can be compromised. Furthermore, there are medical imaging and/or treatment applications for which the information of the atomic numbers of the materials is relied on for the accuracy and performance of the applications. For example, Bremsstrahlung radiation generation is proportional to the square of the atomic number of the material when irradiated by high energy electrons. As such, the volumetric image data can greatly bias the image of bones in Yttrium-90 SPECT theranostic imaging using.

### SUMMARY OF THE INVENTION

Aspects described herein address the above-referenced problems and others.

In one aspect, a system includes a device with memory with spectral volumetric image data generated by a spectrally configured computed tomography scanner including a radiation source and a radiation detector and an image guided system configured to employ the spectral volumetric image data for an image guided procedure.

In another aspect, a computer readable medium is encoded with computer executable instructions, which, when executed by a processor of a computer, cause the processor to: obtain spectral volumetric image data generated by a spectrally configured computed tomography scanner including a radiation source and a radiation detector, and employ the spectral volumetric image data for an image guided procedure.

In another aspect, a method includes receiving spectral volumetric image data generated by a spectrally configured computed tomography scanner including a radiation source and a radiation detector, and utilizing the spectral volumetric image data for an image guided procedure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an example CT imaging system configured for spectral imaging.

FIG. 2 schematically illustrates an example ablation system.

FIG. 3A depicts a non-spectral image showing a tumor and surrounding tissue.

FIG. 3B depicts a virtual monochromatic image reconstructed from spectral projection data and showing the same tumor and surrounding tissue as show in FIG. 3A.

FIG. 4 schematically illustrates an example radiation therapy system.

FIG. 5 schematically illustrates an example SPECT imaging system.

FIG. 6 depicts a reference image of pelvic bone.

FIG. 7 depicts an image of pelvic bone using a Z value estimated for the entire pelvic area for modeling bremsstrahlung radiation.

FIG. 8 depicts an image of pelvic bone using measured Z values for different materials of the pelvic area for modeling bremsstrahlung radiation.

FIG. 9 illustrates an example method in accordance with an embodiment herein.

### DETAILED DESCRIPTION OF EMBODIMENTS

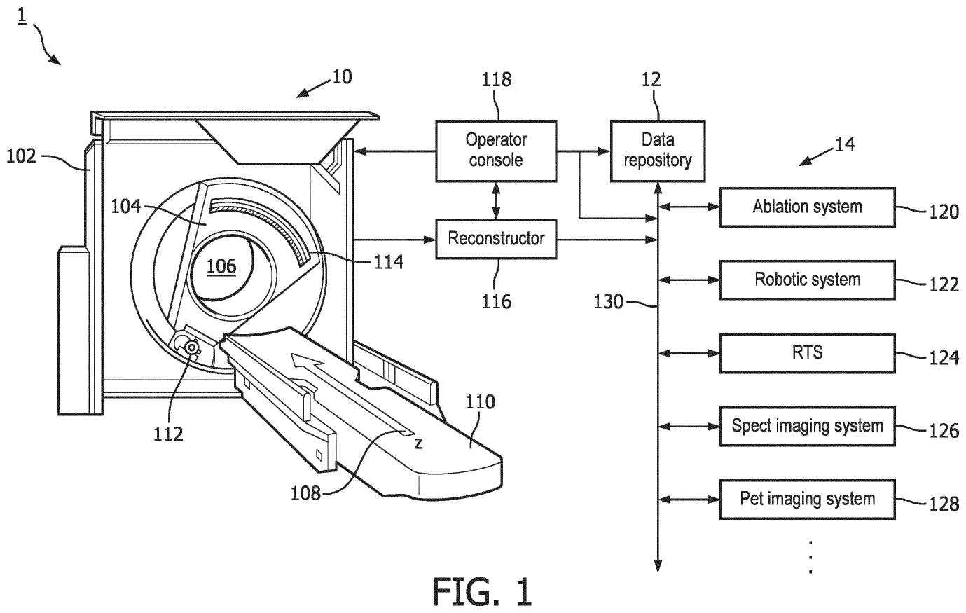

FIG. 1 schematically illustrates a system 1 comprising an imaging system 10, a data repository 12, and at least one image guided system 14.

The illustrated imaging system 10 includes a computed tomography (CT) scanner configured for spectral imaging. The imaging system 100 includes a generally stationary gantry 102 and a rotating gantry 104. The rotating gantry 104 is rotatably supported by the stationary gantry 102 and rotates around an examination region 106 about a longitudinal or z-axis 108. A subject support 110, such as a couch, supports an object or subject in the examination region. The subject support 110 is movable in coordination with performing an imaging procedure so as to guide the subject or object with respect to the examination region 106 for loading, scanning, and/or unloading the subject or object. A radiation source 112, such as an x-ray tube, is rotatably supported by the rotating gantry 104. The radiation source 112 rotates with the rotating gantry 104 and emits X-ray radiation that traverses the examination region 106. In the illustrated embodiment, the radiation source 112 is a single x-ray tube configured to emit broadband (polychromatic) radiation for a single selected peak emission voltage (kVp) of interest (i.e. the energy spectrum at that kVp). In another instance, the radiation source 112 is configured to switch between at least two different emission voltages (e.g., 70 keV, 100 keV, etc.) during scanning. In yet another instance, the radiation source 112 includes two or more x-ray tubes angular offset on the rotating gantry 104 with each configured to emit radiation with a different mean energy spectrum. U.S. Pat. No. 8,442,184 B2 describes a system with kVp switching and multiple x-ray tubes, and is incorporated herein by reference in its entirety.

A radiation spectrum sensitive detector array 114 subtends an angular arc opposite the radiation source 112 across the examination region 106. The detector array 114 includes one or more rows of detectors that arranged with respect to each other along the z-axis 108 direction and detects radiation traversing the examination region 106. In the illustrated embodiment, the detector array 214 includes an energy-resolving detector such as a multi-layer scintillator/photo-sensor detector (e.g., U.S. Pat. No. 7,968,853 B2, which is incorporated herein by reference in its entirety) and/or a photon counting (direct conversion) detector (e.g., WO2009072056A2, which is incorporated herein by reference in its entirety). With an energy-resolving detector, the radiation source 112 includes the broadband, kVp switching and/or multiple X-ray tube radiation source 112. In another instance, the detector array 114 includes a non-energy-resolving detector, and the radiation source 112 includes the kVp switching and/or the multiple X-ray tube radiation source 112. The detector array 114 generates spectral projection data (line integrals) indicative of the different energies.

A reconstructor 116 reconstructs the spectral projection data with multiple different reconstruction algorithms, including a spectral reconstruction algorithm(s) and a non-spectral reconstruction algorithm(s). The non-spectral reconstruction algorithm(s) produces conventional broadband (non-spectral) volumetric image data, e.g., by combing the spectral projection data and reconstructing the combined volumetric image data. The spectral reconstruction algorithm(s) produces basis volumetric image data, e.g., first basis volumetric image data, second basis volumetric image data, . . . , Nth basis volumetric image data. For example, for dual energy, the reconstructor 116 can generate a photo-electric effect and Compton scatter volumetric image data sets, mono-energetic/monochrome volumetric image data sets (e.g., 40 keV and 100 keV), calcium and iodine volumetric image data sets, bone and soft tissue volumetric image data sets, etc. Other data sets include effective Z (atomic number), k-edge, etc. spectral volumetric image data sets.

An operator console 118 allows an operator to control an operation of the system 10. This includes selecting an imaging acquisition protocol (e.g., multi-energy), selecting a reconstruction algorithm (e.g., multi-energy), invoking scanning, etc. The operator console 118 includes an output device(s) such as a display monitor, a filmer, etc., and an input device(s) such as a mouse, keyboard, etc. The projection data and/or volumetric image data can be stored in a memory device of the imaging system 10, such as a memory device of the console 118 and/or a memory device of the reconstructor 116. In the illustrated embodiment, the data repository 12 also can store the projection data and/or volumetric image data. The data repository 12 can also store data generated by other systems, such as other imaging systems. Examples of a suitable data repository 12 includes, but is not limited to, a radiology information system (RIS), a picture and archiving system (PACS), a hospital information system (HIS), etc.), an electronic medical record (EMR), etc.

The at least one image guided system 14 includes one or more of an ablation system 120, a robotic system 122, a radiation therapy system (RTS) 124, a single photon emission computed tomography (SPECT) imaging system 126, and a positron emission computed tomography (PET) imaging system 128, etc. As described in greater detail below, the at least one image guided system 14 utilizes spectral volumetric image data, e.g., from the imaging system 10 and/or data repository 12 via a communication channel 130 such as a wire and/or wireless network, a direct connection, etc., to improve features such as tumor ablation, an image guided robotic procedure, radiation therapy, SPECT scanning, PET scanning, etc., relative to a configuration in which the at least one image guided system 14 utilizes non-spectral volumetric image data for these same features.

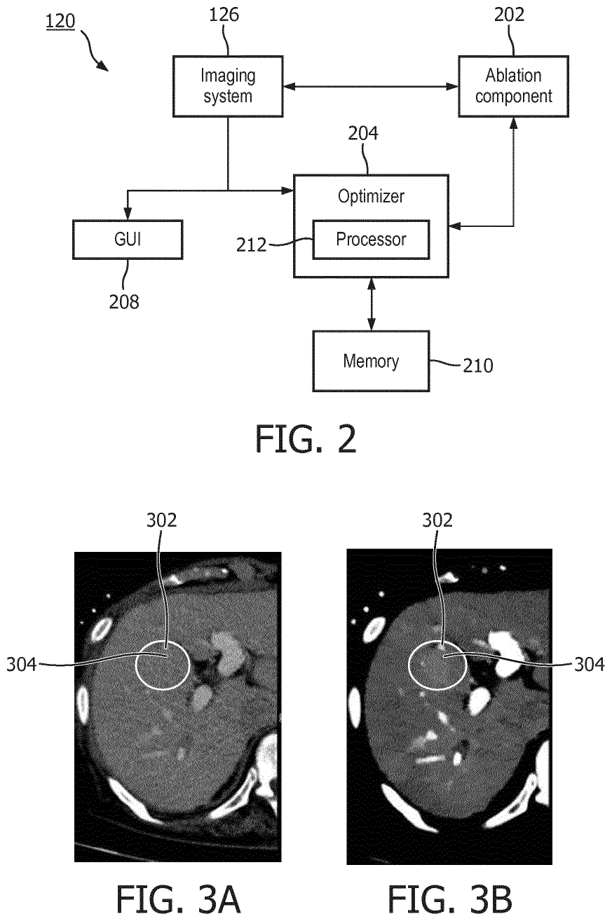

FIG. 2 shows an example of the ablation system 120. In this example, the ablation system 120 includes a radio frequency (RF) ablation system. Examples of suitable ablation systems are described in US 2010/0063496 A1, filed Jul. 15, 2009, and entitled “RF Ablation Planner,” with is incorporated herein by reference in its entirety, U.S. Pat. No. 8,267,927 B2, filed Feb. 22, 2010, and entitled “Advanced Ablation Planning,” which is incorporated herein by reference in its entirety, and/or other ablation system(s). For explanatory purposes, the following discussion is in relation to an ablation system similar to the one described in US 2010/0063496 A1.

The RF ablation system 120 is configured to facilitate generating a plan for performing one or more ablation protocols to treat a tumor mass or lesion in a patient. An example plan includes quantitative information such as target positions and orientations for each ablation. It may also identify an entry point or points on an outside of a body that lead to the target(s). The ablation plan may ensure all areas of the tumor are covered, and reports the number of ablations required for complete ablation using a particular probe. The plan can be carried out using a robot and/or by using registered image guidance, such as by quantitatively tracking the ablation probe.

The illustrated RF ablation system 120 includes an ablation component 202 operatively connected to an optimizer 204 and the imaging system 126. The ablation component 202, in one embodiment, includes at least a power source, a radio frequency generator, a probe operatively coupled thereto, and/or other suitable element(s) to facilitate inserting the probe into a tumor mass and heating the mass to a temperature sufficient to kill tumor cells (e.g., ˜50 degrees Celsius) within a region relative to the probe tip. The ablation component 202 alternatively, or additionally, includes a high-intensity focused ultrasound component (HIFU), which ablates tissue in a particular region through the use of mechanical vibration and/or heating properties of ultrasound.

The optimizer 204 includes a processor 212 that segments objects such as a tumor, lesion, organ, critical region, etc. automatically using algorithms and/or semi-automatically with user input. For tumor/soft tissue discrimination, the processor 212 segments using lower energy spectral volumetric image data. For example, in one instance, the processor 212 processes a 40 keV virtual mono-energic image. FIG. 3A shows contrast between tumor tissue 302 and surrounding tissue 304 for an image generated with non-spectral volumetric image data, and FIG. 3B shows contrast between the same tumor tissue 302 and the same surrounding tissue 304 for an image generated with the 40 keV virtual mono-energic image. These images show greater contrast resolution in FIG. 3B. The particular energy level can be lower or higher, and based on a default, a user preference, an optimization algorithm, etc., and may include in one (as shown) or more images at one (as shown) or more energy levels.

For tumor ablation, using an improved tumor to soft tissue contrast in relatively low energy level spectral volumetric image data can help the definition of the planned target volume (PTV) for the ablation planning. Also, different organs/structures in the patient may have the optimal contrast and delineation in different energy level images. Therefore, multiple energy level spectral volumetric image data may be used to optimize the planning, so that the PTV identification for multiple tumors can be optimized, the line of insertion can be optimized to avoid certain organs/structures, etc. Since the images at different energy levels are intrinsically co-registered, the tumor/organ/structure delineation optimally performed at different energy level images can be simply overlaid into one planning image without worry about registration.

The segmentation produces a description of the volumetric regions associated with the specific objects. A volume may be visually presented via a graphical user interface 208 (GUI). The volume may be ‘grown’ by a desired distance so that the tumor plus margin are included in the resulting volume. The word ‘tumor,’ as used herein, particularly regarding optimization, includes a PTV, which covers a specified tumor plus margin that together are intended for full coverage. Processing tools enable a user to set a margin, whereupon a new PTV is defined. The processor 212 analyzes information associated with the PTV, particularly the dimensions, and for a given ablation probe defines a set of ablation positions with orientations.

In one example, the processor 212 identifies the fewest number of ablations possible that cover the PTV. In another example, the processor 212 identifies the ablation positions with orientations that spares the most healthy tissue (i.e. minimizes collateral damage). In another example, additional object volumes are segmented that denote ‘critical regions’ of tissue or bone that are not to be ablated, and the processor 212 attempts to generate either the fewest ablations or minimize collateral damage, while also avoiding these regions. In another example, the processor 212 produces unablated areas, whereupon the user is alerted and the regions can be displayed on the GUI 208.

Entry angles and/or one or more entry points on a patient's skin can be defined. In one embodiment, a ray marching protocol is employed to determine an entry point. The voxels of the volumetric image data are labeled as either ‘free’ or ‘critical region’, for example in a binary volume. A ray marching algorithm, such as the one introduced by Perlin, “Hypertexture”, Computer Graphics, vol. 23, issue 3, pp. 253-261, 1989), can be employed to identify locations on the skin that permit insertion of a probe into the PTV along a path that does not travel through a sensitive or critical region such as bones. Intuitively, this is similar to setting a light at the center of the tumor, having the critical regions (e.g., solid masses such as bone or the like) block the light, and identifying points where the light reaches the skin.

A ray of light is “marched” from the center of mass (centroid) of the PTV in a linear ‘ray’ through the 3D image until one of three situations occurs: 1) The ray reaches the edge of the image volume, whereupon it restarts at a new orientation from the center of the PTV; 2) The ray reaches the skin or another location approved as an entry point, whereupon the x,y,z location and ray orientation are noted. This is a potential entry point, which may be shown graphically or stored in a list for selection or may be evaluated to determine the number of ablations required for coverage from this angle, or 3) The ray reaches a voxel that is labeled ‘critical region’, whereupon a new ray is begun with a new orientation from the center of the PTV. This procedure continues until all desired angles are evaluated.

The ablation component 202 is utilized to ablate the tumor(s) based on the ablation plan. In general, the ablation system 120 (as well as the robotic medical system 122, the radiation therapy system 124, the SPECT imaging system 126, and/or the PET imaging system 128 of FIG. 1) can utilize spectral volumetric image data in which tumor contrast in soft tissue is a highest and/or multiple spectral images at different energy levels in which different organs/structures of interest have the best contrast/delineation in different images to improve the planning of ablation, as well as robot guided medical, and/or radiation therapy procedures applications, e.g., for tumor and/or critical organ (e.g., spinal cord, eye, genitals, etc.) identification, delineation, the identification of planned target volume, radiation beam path and delivery scheme, etc.

An example of an image guided robotic procedure is discussed in Won et al., “Validation of a CT-guided intervention robot for biopsy and radiofrequency ablation: experimental study with and abdominal phantom,” Diagn Interv Radiol, DOI 10.5152/dir.2017.16422, March 2017. Another robotic example is described in U.S. Pat. No. 6,785,572 B2, filed Nov. 21, 2001, and entitled “Tactile feedback and display in a CT image guided robotic system for interventional procedures,” which is incorporate herein by reference in its entirety, U.S. Pat. No. 5,817,105 A1, filed May 13, 1997, and entitled “Image-guided surgery system,” which is incorporate herein by reference in its entirety, and/or other examples.

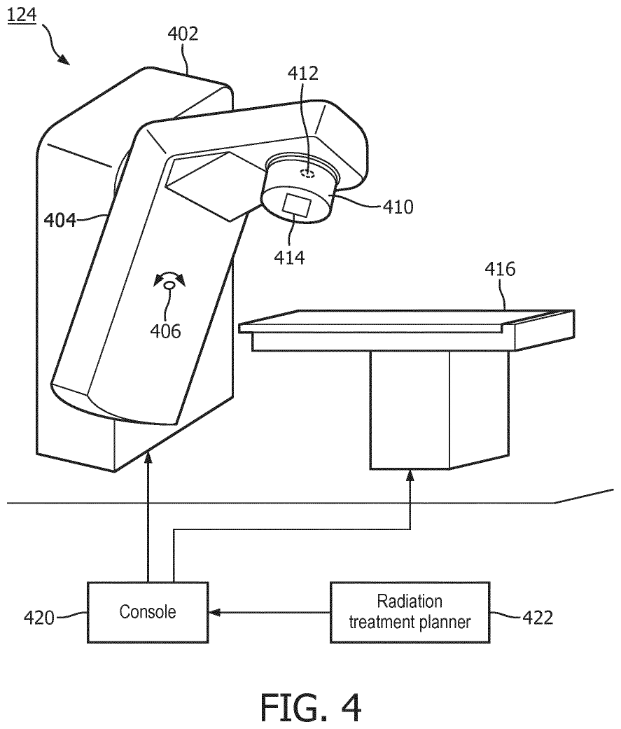

FIG. 4 shows an example of the radiation therapy system 124.

In this example, the radiation therapy system 124 is a linear accelerator, or linac. The radiation therapy system 124 includes a stationary gantry 402 and a rotating gantry 404, which is rotatably attached to the stationary gantry 402. The rotating gantry 404 rotates (e.g., 180°, etc.) with respect to a rotation axis 406 about a treatment region 408. The stationary gantry 402 includes a treatment head 410 with a therapy (e.g., a megavolt (MV) radiation source 412 that delivers treatment radiation and a collimator 414 (e.g., a multi-leaf collimator) that can shape the radiation fields that exit the treatment head 410 into arbitrary shapes.

A subject support 415, such as a couch, supports a portion of a subject in the treatment region 408. A console 420 is configured to the system based on a plan to deliver of treatment radiation by the megavolt radiation source 412 during a treatment. A radiation treatment planner 422 creates radiation treatment. The radiation treatment planner 422 can segment a lesion and identify radiation sensitive tissue with the one or more virtual monochromatic images, identify a planned target volume with the one or more virtual monochromatic images, and/or determine a radiation beam path and delivery scheme with the one or more virtual monochromatic images. Again, the spectral volumetric image data which provides the best contrast/delineation for a particular aspect is utilized.

Another example of an image guided radiation therapy is described in U.S. Pat. No. 9,262,590 B2, filed Jul. 22, 2009, and entitled “Prospective adaptive radiation therapy planning,” U.S. Pat. No. 9,020,234 B2, filed Jul. 22, 2009, and entitled “Contour delineation for radiation therapy planning with real-time contour segment impact rendering,” U.S. Pat. No. 7,596,207 B2, filed Jul. 22, 2009, and entitled “Method of accounting for tumor motion in radiotherapy treatment,” and U.S. Pat. No. 7,708,682 B2, filed Sep. 10, 2004, and entitled “Method and device for planning a radiation therapy,” all of which are incorporated herein by reference in their entireties. Other examples are also contemplated herein.

With radiation therapy, the spectral volumetric image data also allows more accurate estimation of the electron density of the patient body, and therefore, enable more accurate dose simulation, beam planning, and dose calculation in radiation therapy. An example approach includes first reconstructing a virtual mono-energetic spectral image, calculating the electron density map/image from the CT spectral volumetric image data, and then using the calculated electron density map for dose simulation and beam planning, as well as dose calculation. An example of using electron density for dose simulation, beam planning, and/or dose delivery calculation is described in Skrzynski et al., “Computed tomography as a source of electron density information for radiation treatment planning,” Strahlenther Onkol. 2010 June; 186(6):327-33. doi: 10.1007/s00066-010-2086-5.

For calculating the electron density map with spectral volumetric image data for at least two basis materials or high/low energy, the attenuation coefficient of a material (μ(E)) can be approximated by a linear combination of two basis materials μ(E)=b₁μ₁(E)+b₂μ₂(E), where μ₁(E) and μ₂(E) are the attenuation coefficients of the two basis materials and b₁ and b₂ are the basis material coefficients. After solving b₁ and b₂ (e.g., simultaneous equations), the electron density (ρₑ) can be determined through ρₑ=b₁ρ₁+b₂ρ₂, where ρ₁ and ρ₂ are the electron densities of the two basis materials. The electron density map can alternatively be determined otherwise using the spectral volumetric image data.

The image used for the tumor/target identification and delineation can be different from and/or the same as the spectral image used to generate the electron density. For example, the spectral image for tumor/target identification and delineation can be from lower energy images in which tumor to soft tissue contrast is maximal, and the spectral image for the electron density can be from higher energy level images.

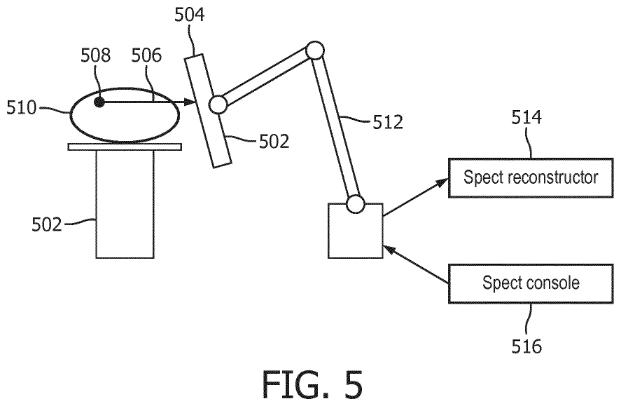

FIG. 5 illustrates an example of the SPECT imaging system 126.

The SPECT imaging system 126 includes a patient support 502 and one or more gamma cameras 504. The one or more gamma cameras 504 detect radiation (e.g., bremsstrahlung photons 506, gamma radiation, etc.) emitted from a radioactive material and/or substance 508 within an objector subject 510. In this example, an articulating arm 512 moves the gamma camera 504 around the objector subject 510. ASPECT reconstructor 514 reconstructs the projections and produces volumetric data. A SPECT console 516 allows a user to control the SPECT scanner 126.

In this example, the SPECT imaging system 126 is configured for Yttrium-90 (⁹⁰Y) theranostic imaging. Generally, β-particle emission from ⁹⁰Y produces bremsstrahlung photons, which can be detected scintigraphically. The ⁹⁰Y bremsstrahlung photons are generated when the high-energy l-particle (i.e., electron) is emitted from the ⁹⁰Y nucleus and then slows (i.e., it loses its kinetic energy) while interacting with adjacent atoms. As the electron slows down, its kinetic energy is converted into the continuous energy spectrum of both primary and scattered photons, i.e. bremsstrahlung radiation.

In one instance, the SPECT imaging system 126 utilizes a reconstruction algorithm that includes a tissue-dependent probability term in the system matrix, i.e., projector/backprojector, to model the bremsstrahlung spectra produced in each voxel as a bone-volume fraction (BVF) weighted mixture of the bone-only and tissue-only spectra. The SPECT imaging system 126 employs atomic number (Z) spectral volumetric image data (e.g., a Z-image) to determine the BVF of each voxel. In general, the Z-image includes an average atomic number of each voxel. Using this measured atomic number provides accurate values for the modeling with improved results, relative to a configuration in which the SPECT imaging system 126 instead uses an estimate from non-spectral CT data.

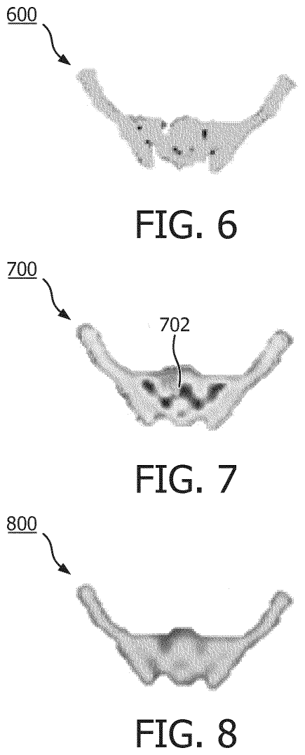

By way of example, FIG. 6 shows a reference (“true”) image 600 of pelvic bone. FIG. 7 shows an image 700 in which Z values for modeling are estimated by segmenting bone from the rest of the tissues in non-spectral CT volumetric image data, assigning an average Z value to all the bone, and then modeling bremsstrahlung with this global average. The image 700 includes non-uniformity and significantly higher values in the cortical bone region 702, relative to the true image 600. FIG. 8 shows an image 800 generated using the approached described herein, which uses measured Z values of bones, marrows, soft tissues, etc. from the atomic number (Z) spectral volumetric image data to model the bremsstrahlung radiation differently for the different body tissues. In this example, image 800, relative to image 700, has improved uniformity and reduced quantitative error.

An example of modeling bremsstrahlung spectra with non-spectral CT volumetric image data in connection with SPECT ⁹⁰Y theranostic imaging is discussed in Wright et al, “Theranostic imaging of Yttrium-90,” BioMed Research International, Vol 2015, Article ID 481279, 2015. Another example of modeling bremsstrahlung spectra with non-spectral CT volumetric image data in connection with SPECT ⁹⁰Y theranostic imaging is discussed in Lim et al., “Y-90 SPECT maximum likelihood image reconstruction with a new model for tissue-dependent bremsstrahlung procedure,” (Abstract), J Nucl Med, vol 58, no. supplement 1, 746, May 1, 2017.

Using the Z-image directly for bremsstrahlung modeling (like in FIG. 8) and not assigning an estimated Z number (like in FIG. 7) to bones mitigates errors in bones and is well-suited for the heterogeneity in bone structures. Moreover, non-spectral CT volumetric image data cannot differentiate materials with different high Z-numbers, such as calcium and iodine, unlike the atomic number (Z) spectral volumetric image data. As such, using atomic number (Z) spectral volumetric image data can improve theranostic imaging when the spectral volumetric image data includes contrast, medical inserts, etc.

The atomic number (Z) spectral volumetric image data can alternatively, or additionally, be used in other applications where an accuracy of the imaging is dependent on an accuracy of the material atomic number information.

The SPECT imaging system 126, and/or the PET imaging system 128 can utilize the virtual mono-energetic spectral volumetric image data, which allows for a more accurate estimation of the linear attenuation coefficients of tissue in patient body, to improve CT-based attenuation correction in PET/CT and/or SPECT/CT. An example of such a correction is described in U.S. Pat. No. 9,420,974 B2, filed May 29, 2009, and entitled “Method and apparatus for attenuation correction,” and US 2011/0123083 A1, filed Jul. 22, 2009, and entitled “Attenuation correction for pet or spect nuclear imaging systems using magnetic resonance spectroscopic image data,” both of which are incorporated herein by reference in their entireties. Other examples are also contemplated herein.

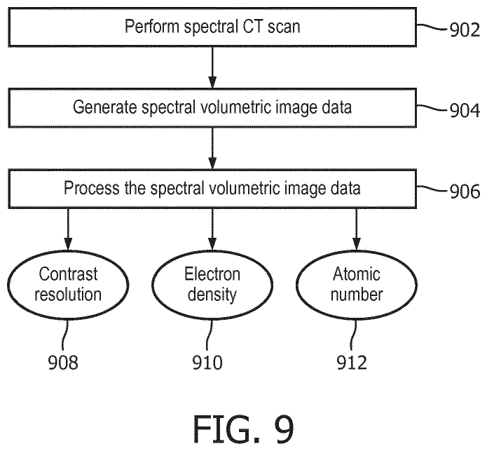

FIG. 9 illustrates an example method in accordance with an embodiment(s) described herein.

It is to be appreciated that the ordering of the acts in the method is not limiting. As such, other orderings are contemplated herein. In addition, one or more acts may be omitted and/or one or more additional acts may be included.

At 902, a spectral CT scan is performed.

At 904, spectral volumetric image data is reconstructed.

At 906, spectral volumetric image data is processed for one or more of improving contrast resolution 908, electron density distribution estimation 910, and atomic number estimation 912, as described herein and/or otherwise.

The above may be implemented by way of computer readable instructions, encoded or embedded on computer readable storage medium (which excludes transitory medium), which, when executed by a computer processor(s) (e.g., central processing unit (cpu), microprocessor, etc.), cause the processor(s) to carry out acts described herein. Additionally, or alternatively, at least one of the computer readable instructions is carried by a signal, carrier wave or other transitory medium, which is not computer readable storage medium.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A system, comprising:
a device with memory including spectral volumetric image data generated by a spectrally configured computed tomography scanner including a radiation source and a radiation detector; and
an image guided system configured to employ the spectral volumetric image data for an image guided procedure.

2. The system of claim 1, wherein the spectral volumetric image data includes a lower energy image, and the image guided system is configured to segment, from the lower energy image, a lesion in a region of soft tissue having values similar to the lesion.

3. The system of claim 2, wherein the spectral volumetric image data includes one or more virtual monochromatic images, and the image guided system is configured to identify different tissue types in different virtual monochromatic images.

4. The system of claim 2, wherein the image guided system is an ablation system configured to generate and employ an ablation plan to ablate the lesion based at least on the segmentation, wherein the ablation plan includes a planned target volume for the lesion and a line of insertion.

5. (canceled)

6. (canceled)

7. The system of claim 3, wherein the image guided system visually displays the lower energy image superimposed over the one or more virtual monochromatic images.

8. The system of claim 2, wherein the image guided system is a robotic system configured to generate and employ a plan to remove the lesion based on the segmentation.

9-11. (canceled)

12. The system of claim 1, wherein the spectral volumetric image data includes one or more virtual monochromatic images, and the image guided system is a radiation therapy system configured to derive an electron density map from the one or more virtual monochromatic images.

13. The system of claim 12, wherein the radiation therapy system is further configured to employ the electron density map for at least one of radiation dose planning, radiation dose simulation and radiation dose calculation.

14. The system of claim 1, wherein the spectral volumetric image data includes an atomic number image, and the image guided system is a positron emission tomography system or a single photon emission computed tomography system configured to employ the atomic number image for bremsstrahlung radiation modeling for yttrium-90 theranostic imaging.

15. (canceled)

16. (canceled)

17. A computer readable medium encoded with computer executable instructions, where the computer executable instructions, when executed by a processor, causes the processor to:
obtain spectral volumetric image data generated by a spectrally configured computed tomography scanner including a radiation source and a radiation detector; and
employ the spectral volumetric image data for an image guided procedure.

18. The computer readable medium of claim 17, wherein the computer executable instructions, when executed by the processor, further cause the processor to:
segment a lesion in the spectral volumetric image data;
identify different tissue in different energy images of the spectral volumetric image data; and
generate and employ a plan to remove the lesion based on the segmentation.

19. The computer readable medium of claim 17, wherein the computer executable instructions, when executed by the processor, further cause the processor to:
segment a lesion and identify radiation sensitive tissue in the spectral volumetric image data;
identify a planned target volume in the spectral volumetric image data; and
determine a radiation beam path and delivery scheme with the spectral volumetric image data.

20. The computer readable medium of claim 17, wherein the computer executable instructions, when executed by the processor, further cause the processor to:
derive an electron density map from the spectral volumetric image data; and
employ the electron density map for at least one of radiation dose planning, radiation dose simulation and radiation dose calculation.

21. The computer readable medium of claim 17, wherein the computer executable instructions, when executed by the processor, further cause the processor to:
employ an atomic number image of the spectral volumetric image data for bremsstrahlung radiation modeling for yttrium-90 theranostic imaging.

22. The computer readable medium of claim 17, wherein the computer executable instructions, when executed by the processor, further cause the processor to:
utilize the spectral volumetric image data for CT-based attenuation correction in at least one of positron emission or single photon emission computed tomography.

23. A method, comprising:
receiving spectral volumetric image data generated by a spectrally configured computed tomography scanner including a radiation source and a radiation detector; and
utilizing he spectral volumetric image data for an image guided procedure.

24. The method of claim 23, further comprising:
segmenting a lesion in the spectral volumetric image data;
identifying different tissue in different energy images of the spectral volumetric image data; and
generating and employ a plan to remove the lesion based on the segmentation.

25. The method of claim 23, further comprising:
segmenting a lesion and identify radiation sensitive tissue in the spectral volumetric image data;
identifying a planned target volume in the spectral volumetric image data; and
determining a radiation beam path and delivery scheme with the spectral volumetric image data.

26. The method of claim 23, further comprising:
deriving an electron density map from the spectral volumetric image data; and
employing the electron density map for at least one of radiation dose planning, radiation dose simulation and radiation dose calculation.

27. The method of claim 23, further comprising:
employing an atomic number image of the spectral volumetric image data for bremsstrahlung radiation modeling for yttrium-90 theranostic imaging.

28. (canceled)
